(19) United States
(12) Patent Application Publication
(10) Pub. No.: US 2025/0225162 A1
(43) Pub. Date: Jul. 10, 2025

(54) INFORMATION PROCESSING DEVICE, INFORMATION PROCESSING METHOD, AND STORAGE MEDIUM

(71) Applicant: CANON MEDICAL SYSTEMS CORPORATION, Otawara-shi (JP)

(72) Inventors: Yusuke KANO, Edinburgh (GB); Anri YAMAZAKI, Nasushiobara (JP)

(73) Assignee: CANON MEDICAL SYSTEMS CORPORATION, Otawara-shi (JP)

(21) Appl. No.: 19/002,212

(22) Filed: Dec. 26, 2024

(30) Foreign Application Priority Data
Jan. 5, 2024 (JP) 2024-000750

(51) Int. Cl. G06F 16/3329; G16H 15/00

(52) U.S. Cl. CPC G06F 16/33295; G16H 15/00

(57) ABSTRACT

An information processing device of an embodiment includes a processing circuit. The processing circuit acquires a reply from a user. The processing circuit estimate medical treatment information including a plurality of preferences of the user regarding medical treatment on the basis of the reply. The processing circuit determine a response to the reply on the basis of the medical treatment information. The processing circuit outputs the response via an output interface. Furthermore, the processing circuit determines, as the response, at least one of a narrowed response and an approval request to the user.

### CROSS-REFERENCE TO RELATED APPLICATION

Priority is claimed on Japanese Patent Application No. 2024-000750, filed Jan. 5, 2024, the content of which is incorporated herein by reference.

### FIELD

Embodiments disclosed in the present specification and the drawings relate to an information processing device, an information processing method, and a storage medium.

### BACKGROUND

Shared decision-making, where patients and medical professionals discuss treatment goals, preferences, and values and cooperate to find an optimal treatment, has been increasingly important. When artificial intelligence (AI) is used in such situations, assistance to provide information that allows patients and medical professionals to discuss efficiently is more important than assistance to directly provide medical determinations. However, when patients have many preferences that make them worried about or be interested in medical treatment, it takes a lot of time and effort for medical professionals to draw out preferences through dialogue with the patients.

### BRIEF DESCRIPTION OF THE DRAWINGS

FIG. 1 is a diagram which shows an example of a configuration of an information processing system according to an embodiment.

FIG. 2 is a diagram which shows an example of a configuration of an information processing system according to an embodiment.

FIG. 3 is a diagram which shows an example of a configuration of an information processing system according to an embodiment.

FIG. 4 is a diagram which shows an example of a configuration of an information processing device according to an embodiment.

FIG. 5 is a flowchart which shows a flow of a series of processing of a processing circuit according to an embodiment.

FIG. 6 is a diagram which shows an example of preferences.

FIG. 7 is a diagram which shows an example of preferences.

FIG. 8 is a diagram for describing a method of estimating a rating for each preference.

FIG. 9 is a diagram for describing a method of determining a response to be given to a patient.

FIG. 10 is a diagram for describing a method of generating a summary report and a narrowed response.

FIG. 11 is a diagram which shows an example of an online learning algorithm.

FIG. 12 is a diagram for describing a method of generating a summary report.

FIG. 13 is a diagram for describing a method of generating a narrowed response.

FIG. 14 is a diagram which shows another example of a GUI screen of a terminal device.

### DETAILED DESCRIPTION

Hereinafter, an information processing device, an information processing method, and a storage medium according to an embodiment will be described with reference to the drawings.

The information processing device according to an embodiment has a processing circuit. The processing circuit acquires a reply from a user. The processing circuit estimates medical treatment information including a plurality of preferences of the user regarding medical treatment on the basis of the reply. The processing circuit determines a response to the reply on the basis of the medical treatment information. The processing circuit outputs the response via an output interface. The processing circuit further determines, as the response, at least one of a narrowed response, which is a response made to the reply from the user to narrow down the plurality of preferences, and an approval request to the user for each of the plurality of preferences. As a result, it is possible to support efficient questioning of a patient to know preferences of the patient regarding medical treatment without involvement of a medical professional.

### [Configuration of Information Processing System]

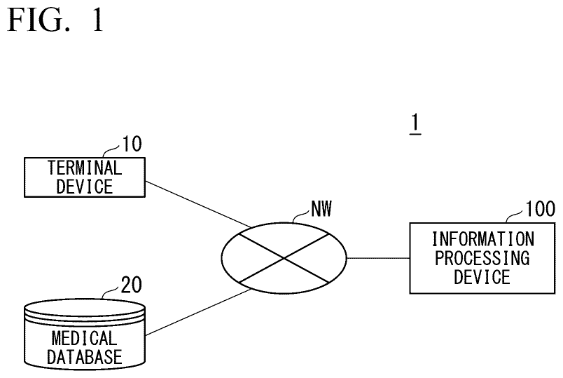

FIG. 1 is a diagram which shows an example of a configuration of an information processing system 1 according to an embodiment. The information processing system 1 includes, for example, a terminal device 10, a medical database 20, and an information processing device 100. The terminal device 10, the medical database 20, and the information processing device 100 are communicatively connected via, for example, a communication network NW.

The communication network NW may refer to a general information and communication network that uses an electrical communication technology. For example, the communication network NW includes telephone communication line networks, optical fiber communication networks, cable communication networks, and satellite communication networks, as well as wireless or wired local area networks (LANs) such as hospital backbone LANs and Internet networks.

The terminal device 10 is, for example, a terminal device such as a personal computer, a tablet terminal, or a mobile phone, and is used by patients and medical professionals. Medical professionals are typically doctors, but may also be nurses or other people involved in medical treatment.

For example, a patient may input his or her replies to questions into the terminal device 10 by touching or using speech. In addition, a medical professional may ask the patient a question orally, listen to a reply to the question from the patient, and input a result of the listening into the terminal device 10. The questions include matters related to medical treatment.

In the present embodiment, “medical treatment” may include not only treatment such as surgery and medication, but also medical examinations leading up to or after treatment, and any other medical procedures.

The terminal device 10 transmits information entered by a patient or a medical professional to the information processing device 100 via the communication network NW, or receives information from the information processing device 100.

In particular, the terminal device 10 displays, as a graphical user interface (GUI), additional questions (hereinafter referred to as narrowed responses) that are asked as a response to a reply of the patient to narrow down one or a plurality of preferences from among many matters related to medical treatment, based on the information received from the information processing device 100.

Preferences are typically matters that the patient is worried about or interested in regarding medical treatment, but the present invention is not limited thereto. For example, preferences may be matters that the patient feels apply to himself or herself regarding medical treatment, matters that the patient agrees with, matters that the patient feels have a high affinity with, matters that make sense, matters that seem plausible, matters that give the patient little resistance, and the like, in addition to or instead of the matters that the patient is worried about or interested in regarding medical treatment.

For example, when the “medical treatment” is “disease differentiation for colds,” the patient is questioned about several symptoms such as sore throat, cough, phlegm, and fever as part of a medical interview. In such a case, symptoms that the patient replies with as being applicable to himself or herself (in other words, symptoms that the patient is aware of) among the plurality of symptoms questioned about serve as preferences. In this manner, preferences may be matters selected based on various emotions or inner thoughts of the patient, such as worries, interests, zests, and tastes.

Furthermore, the terminal device 10 displays, as a GUI, a summary report customized by a user based on replies from the patient to a plurality of questions including a narrowed response.

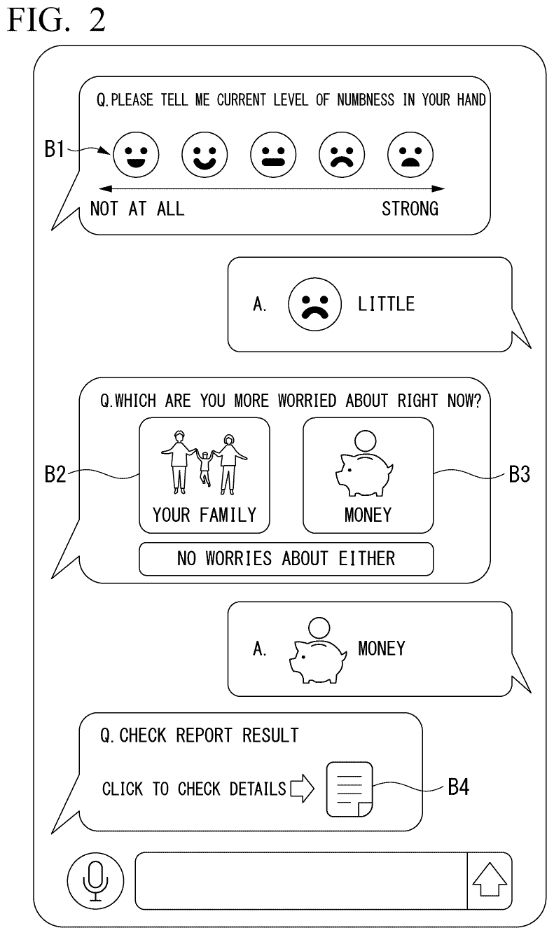

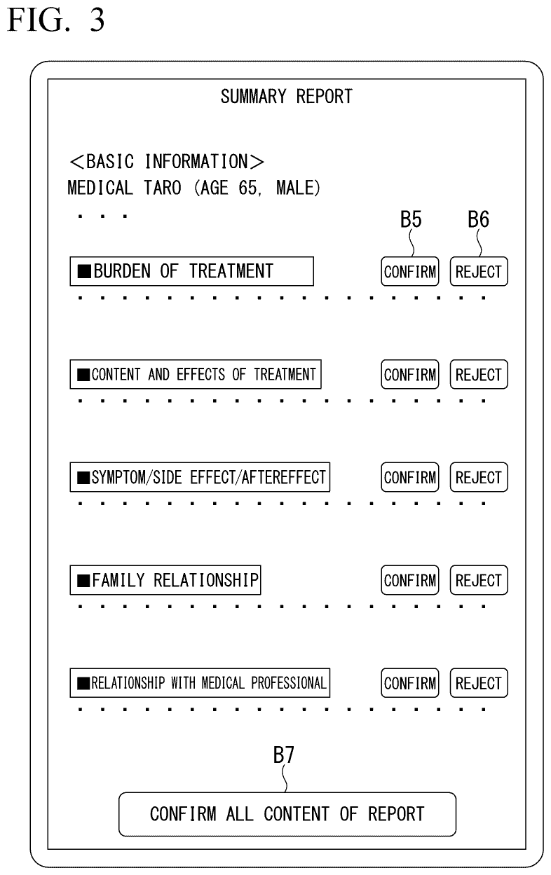

FIGS. 2 and 3 are diagrams which show an example of a GUI screen of the terminal device 10. FIG. 2 shows an example in which a narrowed response is further displayed in response to a reply to a question about a certain medical treatment from the patient.

As shown in the FIGS. 2 and 3, for example, it is assumed that a question such as “Please tell me a current level of numbness in your hands” is asked. In such a case, the patient replies to the question by operating an object (an operation button) that allows the patient to select a degree of numbness in stages, as shown in B1. In the shown example, the patient replies with “a little.”

In the present embodiment, a narrowed response is made to such a reply, such as “Which are you more worried about right now, your family or money?” B2 represents an object for selecting “family,” and B3 represents an object for selecting “money.” In the shown example, the patient replies with “money.”

Then, after a series of dialogue consisting of such a reply of the patient and a response to this, a link to a summary report customized by the user on the basis of content of the series of dialogues is displayed. B4 represents an object (an operation button) for accessing the summary report.

For example, when the patient selects the object B4, a summary report like that shown in FIG. 3 is displayed. The summary report displays suggested preferences for the patient, such as “burden of treatment,” “content and effects of treatment,” “symptoms/side effects/aftereffects,” “family relationships,” and “relationships with medical professionals.”

For each of the plurality of suggested preferences, the summary report displays an object B5, which allows the patient to confirm the preference, and an object B6, which allows the patient to reject the preference. Furthermore, the summary report also displays an object B7, which allows the patient to confirm all the suggested preferences at once.

For example, when the patient operates objects B5 to B7, the results are fed back. As a result, questions to the patient from a next time onwards can be made closer to questions related to more appropriate preferences (for example, preferences that make the patient more worried, more interested, or feel apply to himself or herself). A detailed algorithm for the narrowed response and summary report will be described below.

Returning to the description of FIG. 1, the medical database 20 is a database that stores attribute information and medical examination data of patients. The medical database 20 transmits the attribute information and medical examination data of patients to the information processing device 100 via the communication network NW. The medical database 20 may also store data transmitted from the information processing device 100. The medical database 20 may be, for example, a general-purpose server or a cloud server.

The information processing device 100 receives information from the terminal device 10 and the medical database 20 via the communication network NW and processes the received information. For example, the information processing device 100 generates the narrowed response and the summary report described above. The information processing device 100 then transmits the processed information to the terminal device 10 and the medical database 20 via the communication network NW. In addition to or instead of transmitting the processed information to the terminal device 10, the information processing device 100 may transmit the processed information to a dedicated terminal for medical professionals installed in a hospital.

The information processing device 100 may be a single device, or a system in which a plurality of devices connected via a communication network NW operate together. That is, the information processing device 100 may be realized by a plurality of computers (processors) included in a distributed computing system or a cloud computing system. In addition, the information processing device 100 does not necessarily have to be a separate device from the terminal device 10, and may be a device integrated with the terminal device 10.

### [Configuration of Information Processing Device]

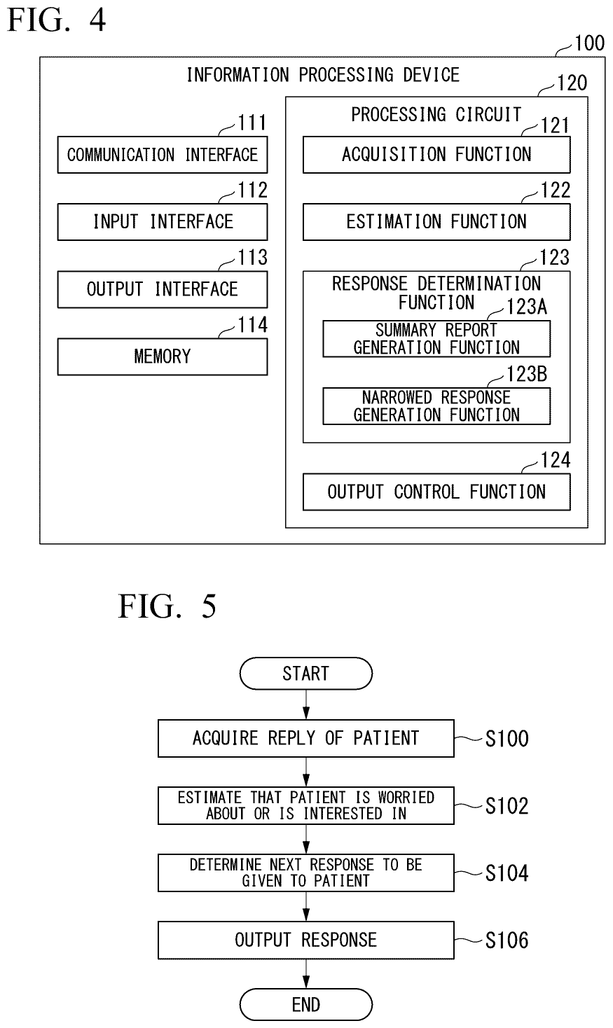

FIG. 4 is a diagram which shows an example of a configuration of the information processing device 100 according to the embodiment. The information processing device 100 includes, for example, a communication interface 111, an input interface 112, an output interface 113, a memory 114, and a processing circuit 120.

The communication interface 111 communicates with external devices via the communication network NW. The external devices include, for example, the terminal device 10 and the medical database 20. The communication interface 111 includes, for example, a network interface card (NIC), an antenna for wireless communication, and the like.

The input interface 112 receives various input operations from an operator, converts the received input operations into electrical signals, and outputs them to the processing circuit 120. For example, the input interface 112 includes a mouse, a keyboard, a trackball, a switch, a button, a joystick, a touch panel, and the like. The input interface 112 may be, for example, a user interface that receives an audio input from a microphone or the like. When the input interface 112 is a touch panel, the input interface 112 may also have a display function of a display 113a included in the output interface 113, which will described below.

In this specification, the input interface 112 is not limited to an interface equipped with physical operating parts such as a mouse and a keyboard. For example, an example of the input interface 112 includes a processing circuit of an electrical signal that receives an electrical signal corresponding to an input operation from an external input device provided separately from a device and outputs this electrical signal to a control circuit.

The output interface 113 includes, for example, a display 113a and a speaker 113b. The display 113a displays various types of information. For example, the display 113a displays an image generated by the processing circuit 120, a GUI for receiving various input operations from an operator, and the like. For example, the display 113a is a liquid crystal display (LCD), a cathode ray tube (CRT) display, an organic electro luminescence (EL) display, or the like. The speaker 113b outputs the information input from the processing circuit 120 using a sound.

The memory 114 is realized, for example, by a semiconductor memory element such as a random access memory (RAM) or a flash memory, a hard disk, or an optical disk. These non-transitory storage media may be realized by other storage devices connected via a communication network NW, such as a network attached storage (NAS) or an external storage server device. The memory 114 may also include a non-transitory storage medium such as a read only memory (ROM) or a register. The memory 114 stores a program executed by the hardware processor of the processing circuit 120, various calculation results by the processing circuit 120, and the like.

The processing circuit 120 includes, for example, an acquisition function 121, an estimation function 122, a response determination function 123, and an output control function 124. The response determination function 123 includes a summary report generation function 123A and a narrowed response generation function 123B.

The processing circuit 120 realizes these functions by, for example, a hardware processor (computer) executing a program stored in the memory 114 (storage circuit). The acquisition function 121 is an example of an “acquisition unit,” the estimation function 122 is an example of an “estimation unit,” the response determination function 123 is an example of a “response determination unit,” and the output control function 124 is an example of an “output control unit.”

The hardware processor in the processing circuit 120 refers to, for example, a circuit (circuitry) such as a central processing unit (CPU), a graphics processing unit (GPU), an application specific integrated circuit (ASIC), a programmable logic device (for example, a simple programmable logic device (SPLD), a complex programmable logic device (CPLD), or a field programmable gate array (FPGA)). Instead of storing the program in the memory 114, the program may be directly incorporated into a circuit of the hardware processor. In this case, the hardware processor realizes the function by reading and executing the program incorporated in the circuit. The program described above may be stored in the memory 114 in advance, or may be stored in a non-transitory storage medium such as a DVD or CD-ROM, and may be installed from the non-transitory storage medium to the memory 114 when the non-transitory storage medium is attached to a drive device (not shown) of the information processing device 100. The hardware processor is not limited to being configured as a single circuit, and may be configured as a single hardware processor by combining a plurality of independent circuits to realize each function. In addition, a plurality of components may be integrated into a single hardware processor to realize each function.

### [Processing Flow of Information Processing Device]

A series of processing by the processing circuit 120 of the information processing device 100 will be described below with reference to a flowchart. FIG. 5 is a flowchart which shows a flow of the series of processing by the processing circuit 120 according to the embodiment.

First, when a patient is asked of a question regarding medical treatment, the acquisition function 121 acquires a reply of a patient to the question (step S100).

For example, the acquisition function 121 may acquire the reply of the patient from the terminal device 10 via the communication interface 111. In addition, when a medical professional such as a doctor of the patient inputs the reply of the patient into the input interface 112, the acquisition function 121 may acquire the reply of the patient from the input interface 112. Furthermore, when the reply of the patient is stored in the memory 114, the acquisition function 121 may acquire the reply of the patient from the memory 114.

When the acquisition function 121 acquires the reply of the patient, it may acquire attribute information of the patient from the medical database 20 via the communication interface 111. The attribute information of the patient may include, for example, age, sex, weight, blood types, vital signs, concurrent diseases, expected complications, epidemiological information, a patient functioning index, a main disease state, overall condition, hereditary characteristics, a medical history, a family history, a medical history, a lifestyle habit, a diseased organ state, a state other than diseased organ, a tumor state, and the like.

Next, the estimation function 122 estimates a plurality of preferences that the patient is worried about, is interested in, or feels apply to the patient regarding medical treatment (step S102). Preferences are an example of “medical treatment information.”

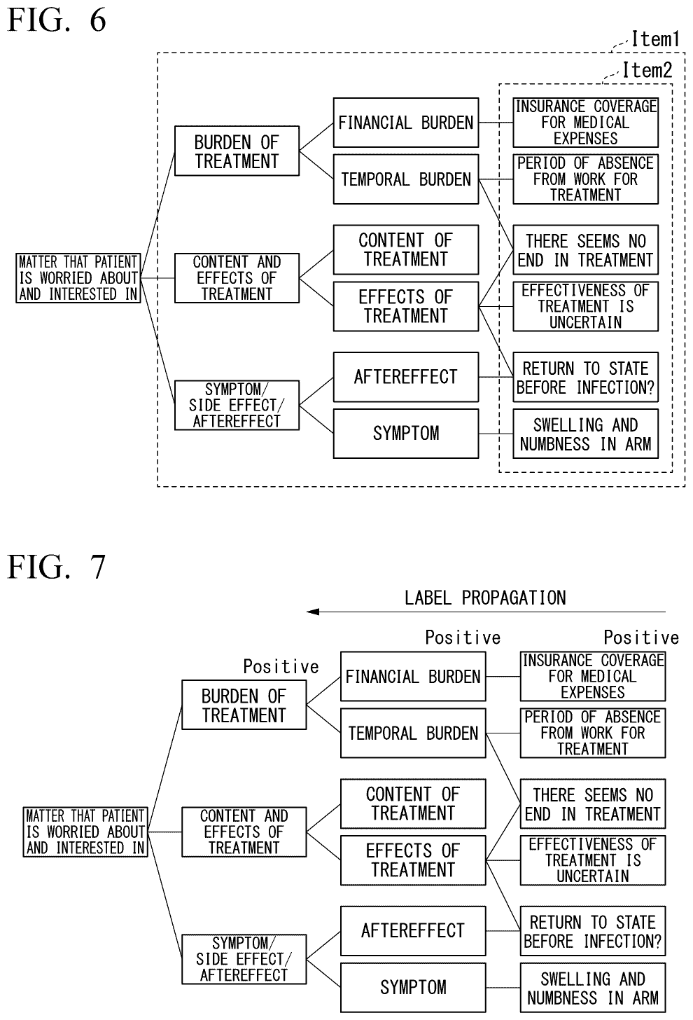

FIGS. 6 and 7 are diagrams which show examples of preferences. As shown in the FIGS. 6 and 7, preferences have a hierarchical structure (stratified). Matters questioned to the patient as the narrowed response described above may cover all preferences, as shown in Item 1 in the FIGS. 6 and 7. On the other hand, the matters included in the summary report may be preferences in a bottom layer, as shown in Item 2 in the FIGS. 6 and 7.

The questions posed as the narrowed response may directly or indirectly correspond to preferences. The summary report includes content for a plurality of preferences that corresponds to ratings for each preference.

A rating is an index that quantifies a degree to which the patient worries about each preference, a degree to which the patient is interested in each preference, and a degree to which the patient feels that each preference applies to himself or herself. The estimation function 122 estimates the ratings of each preference to estimate the preferences. The summary report does not necessarily have to be expressed in natural language (character strings) only, and may include, for example, a graph that quantitatively indicates the ratings, and the like. The ratings are another example of “medical treatment information.” In addition, the ratings are an example of a “predetermined index.”

Furthermore, the preferences may be given a label (Positive in FIGS. 6 and 7) indicating that the patient has given a positive reply in the past. In the optimization of matrix factorization, which will be described below, label propagation is performed from preferences in lower layers to preferences in upper layers.

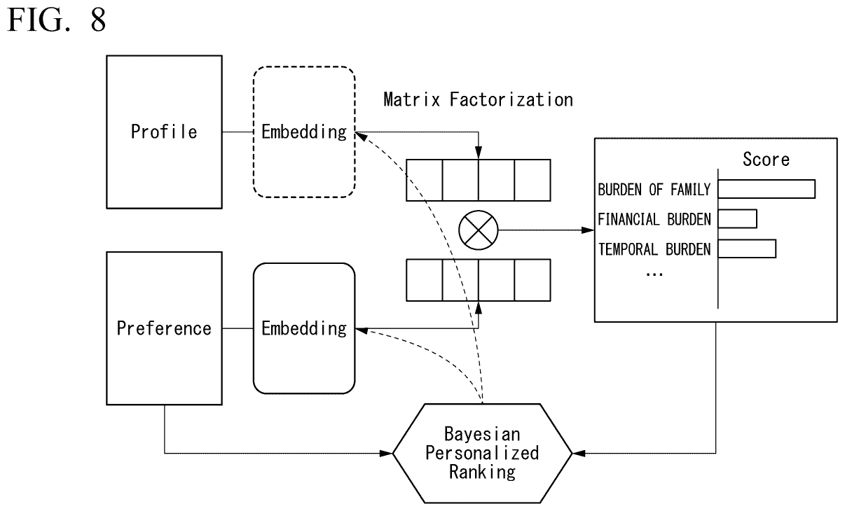

FIG. 8 is a diagram for describing a method of estimating ratings for each preference. As shown in the FIG. 8, for example, the estimation function 122 estimates ratings for the preferences of a target patient using a matrix factorization that has been optimized in advance (pre-trained).

Matrix factorization is a technique in which the patient (user) and preferences are represented as feature vectors of the same length (reduction in dimensionality), and the rating is expressed as an inner product of the feature vectors. The rating estimation using matrix factorization can be represented, for example, by Equations (1) to (3).

$$\begin{matrix}[ Equation 1 ] & \\ {u}_{i} = FCLayers ( {x}_{i} ) & ( 1 )\end{matrix}$$

$$\begin{matrix}[ Equation 2 ] & \\ {p}_{j} = Embedding ( {v}_{j} ) & ( 2 )\end{matrix}$$

$$\begin{matrix}[ Equation 3 ] & \\ {r}_{i , j} = {u}_{i}^{T} {p}_{j} & ( 3 )\end{matrix}$$

Here, uᵢ represents a latent feature vector of an i^{th} patient (user). xᵢ represents an observed feature amount of the i^{th} patient (user). pⱼ represents a latent feature vector of a j^{th} preference. vⱼ represents an index of the j^{th} preference. r_{i,j} represents a rating of the i^{th} patient (user) for the j^{th} preference.

When meta-information of the patient (user) and preferences is available, a cold-start problem can be addressed by using a function (such as fully-connected layers) that converts the meta-information into a feature vector instead of embedding. Here, it is assumed that meta-information of the patient (user) (an observed feature amount of attributes such as an age and gender of the patient) is available, so this meta-information is converted into a feature vector in the fully-connected layers. In addition, since preferences have a hierarchical structure, existing methods that take the hierarchical structure into account may be used when learning a feature vector by embedding.

To optimize matrix factorization (to learn matrix factorization), a loss function based on Bayesian personalized ranking (Bayesian personalized ranking) is used.

Bayesian personalized ranking is a method used for implicit feedback (cases where users do not explicitly provide ratings) such as click history and purchase history, and evaluates only a magnitude correlation between rankings of pairs of preferences.

A loss function based on Bayesian personalized ranking can be represented, for example, by Equations (4) to (6).

$$\begin{matrix}[ Equation 4 ] & \\ Loss = {\sum }_{( u , x , y ) ∈ {𝒟}_{s}} - ln σ ( {r}_{u , x} - {r}_{u , y} ) + {λ}_{Θ} {‖ Θ ‖}^{2} & ( 4 )\end{matrix}$$

$$\begin{matrix}[ Equation 5 ] & \\ {𝒟}_{s} := \{ ( u , x , y ) \vert x , y ∈ {P}_{u}^{+} ∧ y ∈ P \backslash {P}_{u}^{+} \} & ( 5 )\end{matrix}$$

$$\begin{matrix}[ Equation 6 ] & \\ {𝒟}_{s} := \{ ( u , x , y ) \vert x , y ∈ {P}_{u}^{x > y} \} & ( 6 )\end{matrix}$$

Here, Θ represents a parameter to be optimized. λ_{θ} represents a coefficient of a regularization term. P represents a set of all preferences. Pᵤ⁺ represents a set of preferences evaluated by the user as positive examples. Pᵤ^{x>y} represents a set of pairs of preferences compared by the user.

When the feedback is only positive examples, as in Equation (5), preferences not evaluated by the user are sampled as negative examples (negative sampling). When the hierarchical structure of preferences is taken into account, as in Equation (6), a calculation of losses may be limited to only items at the same hierarchical level, as pairwise feedback.

When preferences are labeled, as in FIG. 7 described above, label propagation may be performed to learn a relationship in the hierarchical structure of preferences. That is, a label of the higher-level preference may be determined based on a label of the lower-level preference. For example, it is assumed that a positive label has been given to the lower-level preference such as “regarding insurance coverage of medical expenses.” In this case, “financial burden” and “burden of treatment,” which are higher-level preferences than “regarding insurance coverage of medical expenses,” are also given positive labels. Furthermore, the label of the higher-level preference may be determined according to a ratio of labels of the lower-level preferences. For example, graded implicit feedback may be used for this purpose.

Returning to the description of the flowchart, the response determination function 123 then determines a next response to be given to the patient in response to the reply of the patient on the basis of the estimated preferences and the attribute information of the patient (step S104).

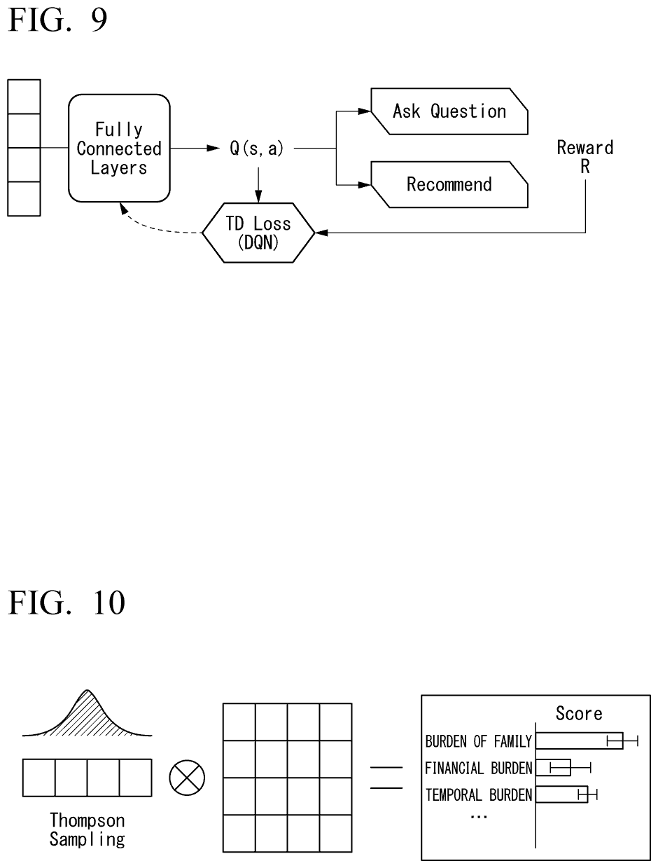

FIG. 9 is a diagram for describing a method for determining a response to be given to the patient. As shown in the FIG. 9, for example, the response determination function 123 may determine a response using a deep Q-network (DQN), which is a representative method of reinforcement learning. The DQN shown in Equations (7) to (9) may be used.

$$\begin{matrix}[ Equation 7 ] & \\ s = Concatenate ( {s}_{his} , {s}_{σ} ) & ( 7 )\end{matrix}$$

$$\begin{matrix}[ Equation 8 ] & \\ Q ( s , a ) = FCLayers ( s ) & ( 8 )\end{matrix}$$

$$\begin{matrix}[ Equation 9 ] & \\ a = arg {max}_{a} Q ( s , a ) & ( 9 )\end{matrix}$$

Sₕᵢₛ is a vector encoding a dialogue history with the patient. s_{σ} represents a confidence interval of preferences. Q(s,a) represents an action value function. s represents a state. a represents an action (Ask Question or Recommend).

A state s in which a Q value is used as an input to a DQN neural network, uses a vector that combines the dialogue history and an estimation confidence of preferences, which are considered to have a strong influence on a determination to ask a question or make a recommendation.

Encoding of the dialogue history is a vector represented as sₕᵢₛ=[+1, −1, −2, +1, +2, 0, 0, 0, 0, 0], where the content of a maximum of 10 turns of dialogue are expressed as follows: questions are expressed as ±1 (a success is +, a failure is −), recommendations are expressed as ±2 (a success is +, a failure is −), and unreached turns are expressed as 0. Here, a turn refers to a series of dialogues in which a question is asked to a patient and the patient replies to the question.

A confidence interval of preferences refers to uncertainty of the ratings for preferences during a dialogue.

Equation (10) represents the loss function used in learning DQN. The loss function may be a TD error based on the Bellman equation, as in a case of a general DQN.

$$\begin{matrix}[ Equation 10 ] & \\ Loss = {( Q ( {s}_{j} , {a}_{j} ) - ( {r}_{j} + γ ( 1 - {d}_{j} ) {max}_{{a}^{′}} Q ( {s}_{j + 1} , {a}^{′} ) ) )}^{2} & ( 10 )\end{matrix}$$

sⱼ represents the state at step j. aⱼ represents the action at step j. rⱼ represents the reward at step j. dⱼ represents a flag indicating whether step j is a terminal (dⱼ is a element of {0,1}). γ represents a discount rate.

The reward r used in reinforcement learning may be set to, for example, any one of the following:
r_{suc}: Approval of summary report (Strongly Positive Reward)
rₚₐᵣₜ: Partial approval of summary report (Positive or Negative Reward depending on a ratio of rejection)
rᵣₑⱼ: Rejection of summary report (Strongly Negative Reward)
r_{quit}: End of dialogue (Strongly Negative Reward)
rₜᵤᵣₙ: Cost consumed each turn (Slightly Negative Reward)

The summary report generation function 123A included in the response determination function 123 generates a summary report as a response to the patient, and the narrowed response generation function 123B included in the response determination function 123 generates a narrowed response as a response to the patient.

FIG. 10 is a diagram for describing a method of generating a summary report and a narrowed response. The summary report generation function 123A and the narrowed response generation function 123B generate a summary report and a narrowed response while adapting them to the reply from the patient obtained during the dialogue. Online learning is performed for this.

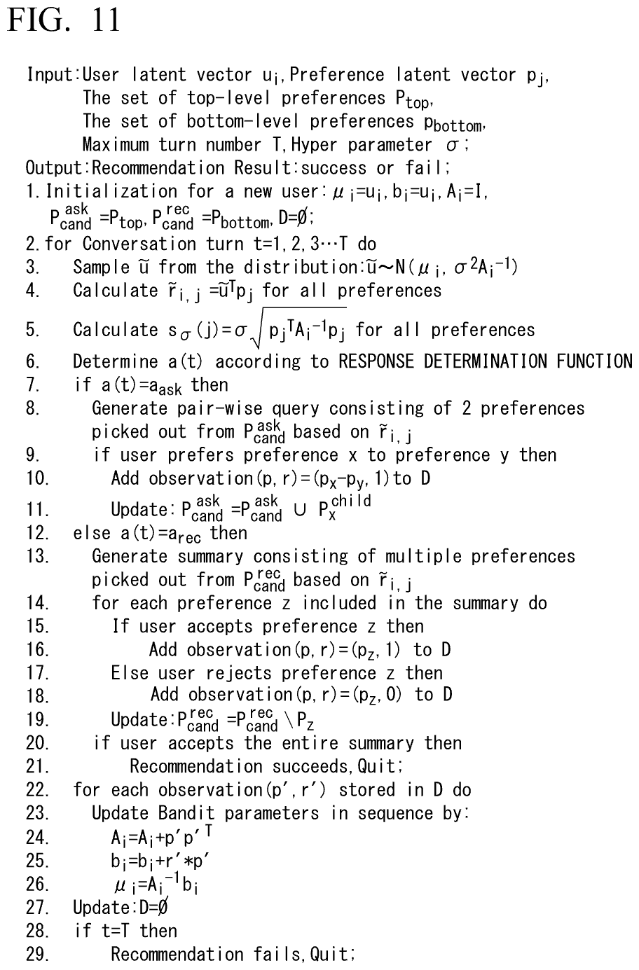

FIG. 11 is a diagram which shows an example of an algorithm of online learning. P_{cand}^{ask} represents preferences of question candidates. P_{cand}^{rec} represents the preferences of recommended candidates. Pₐ represents an a^{th} preference. Pₐ^{child} represents a set of preferences corresponding to a children of Pₐ. Aᵢ and bᵢ represent parameters of a contextual bandit.

The algorithm shown in FIG. 11 is an algorithm that is based on Linear Thompson Sampling (LinTS), a type of a contextual bandit algorithm, and is used to balance the search and utilization of selection of a dialogue-based recommendation system. This algorithm is characterized by sampling a feature vector of a user from a certain probability distribution to estimate preferences of the user while taking into account uncertainty at a beginning of a dialogue.

For example, as shown in lines 3 and 4, a rating adapted to the reply from the user during the dialogue is estimated by using the feature vector of the user sampled from a multivariate normal distribution with a mean vector and a covariance matrix updated during the dialogue on the basis of LinTS.

As shown in a line 1, the feature vector identified by the estimation function 122 is set as an initial value of the mean vector of the multivariate normal distribution. As a result, it is possible to perform an estimation with a certain degree of accuracy even at a beginning of a dialogue, relying on the observed feature amount of the user.

Processing shown in lines 7 to 11 is processing used when the user is asked about two preferences pairwise.

Processing shown in a line 10 is processing used to reflect feedback on the pairwise question in the contextual bandit. Reward is set for a difference between feature vectors of the two preferences.

In questions about preferences, it is desirable to start by asking about the preferences in upper layers, and then dig down to lower layers to ask about preferences that interest the user. Therefore, the preferences P_{cand}^{ask} in question candidates set the preferences in the upper layers as initial values, and the preferences in the lower layers are added to the candidates depending on the reply of the user (a line 11).

Processing shown in lines 12 to 21 is processing for recommending a summary report consisting of a plurality of preferences to the user.

Since feedback is obtained simultaneously for a plurality of preferences, an absolute reward is set for each preference. Here, since it is assumed that preferences included in the summary report are only bottom-level preferences, the preferences P_{cand}^{rec} of recommended candidates have the bottom-level preferences as initial values, and preferences that have already been recommended are removed from the candidates as needed (line 19).

Parameter update of Contextual Bandit in lines 22 to 26 is the same as an LinTS algorithm.

Lines 8 and 13 are parts that use the rating estimation values r(˜)_{i,j} to determine preferences to ask about and preferences to recommend. There are various methods to determine these preferences.

Typically, among a plurality of preferences, preferences may be adopted in descending order of the rating estimation value r(˜)_{i,j}. When preferences with small ratings are also important, preferences may be adopted in descending order of an absolute value of the rating estimation value r(˜)_{i,j}.

Moreover, for pairwise questions, a comparison of the top two preferences of the rating estimation value r(˜)_{i,j} is not necessarily optimal for narrowing the feature vector of the user. Factors other than the rating estimation value r(˜)_{i,j} may also be taken into account.

This algorithm can be extended to cases where the same user uses it a plurality of times. In this case, it is possible to introduce uncertainty again into the feature vector of the user by taking into account that values of the user change over time. Specifically, uncertainty may be introduced again by setting a previous feature vector of the user as the initial value in a line 1 and adjusting a covariance matrix Aᵢ⁻¹ or a value of a hyperparameter σ that controls the uncertainty.

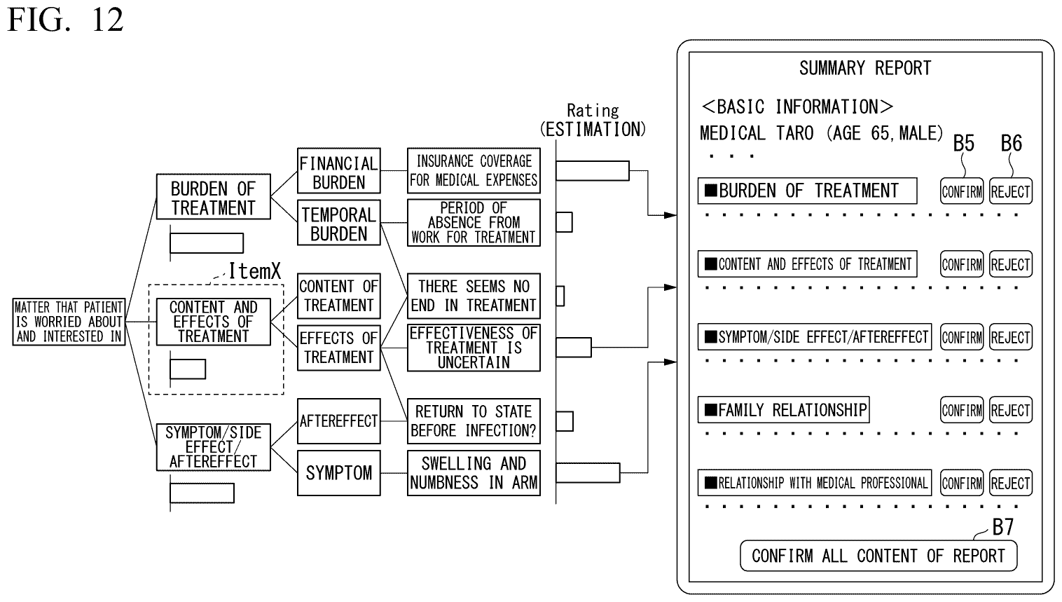

FIG. 12 is a diagram for describing a method for generating a summary report. As shown in the FIG. 12, the summary report generation function 123A may preferentially include preferences with higher ratings among the preferences in the bottom layer in the summary report. In addition, the summary report generation function 123A may include preferences with lower ratings in the summary report.

Furthermore, the summary report generation function 123A may determine the categories included in the summary report based on the ratings, based on the hierarchical structure. For example, as shown in Item X, when a rating of a preference in an upper layer is lower than ratings of other preferences in the same upper layer, the preference with the lower rating in the upper layer and preferences that are present below the preferences in the upper layer may be excluded from the summary report as one category.

As described in FIG. 3, the summary report may allow approval or rejection of preferences as a whole, or approval or rejection of a preference-by-preference basis. Furthermore, the summary report may allow approval or rejection on a category-by-categories basis, and rejected preferences may be able to be modified by the user.

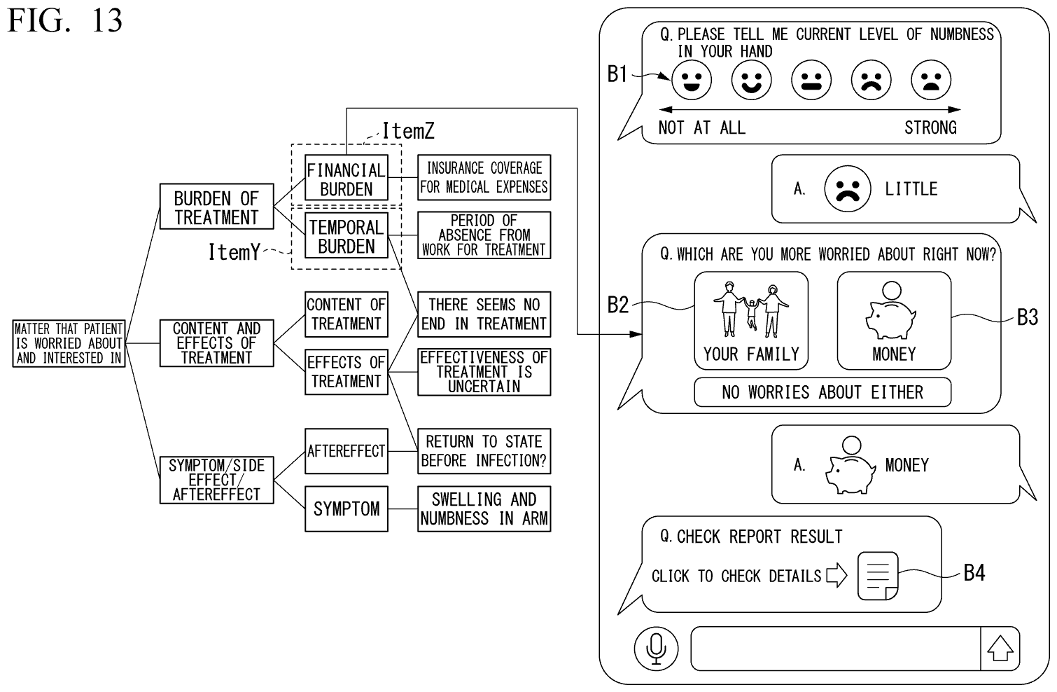

FIG. 13 is a diagram for describing how to generate a narrowed response. Like Item Y and Item Z in FIG. 13, the narrowed response generation function 123B may select any two preferences when a question posed to the patient as a narrowed response is “comparison of two preferences.” The narrowed response generation function 123B may select two preferences at the same hierarchical level or two preferences with the same parent preferences by taking the hierarchical structure into account.

In addition to “comparison of two preferences,” the narrowed response generation function 123B may also generate “Yes or No for one preference,” “rating for one preference,” “reordering of a plurality of preferences,” “a plurality of selections from a plurality of preferences,” and the like as the narrowed responses.

Returning to the description of the flowchart, the output control function 124 then outputs the determined response (summary report or narrowed response) (step S106).

For example, the output control function 124 transmits the determined response to the terminal device 10 via the communication interface 111. As a result, a narrowed response as shown in FIG. 2 or a summary report as shown in FIG. 3 is displayed on the screen of the terminal device 10. The output control function 124 may also display the determined response on the display 113a. As a result, the processing of this flowchart ends.

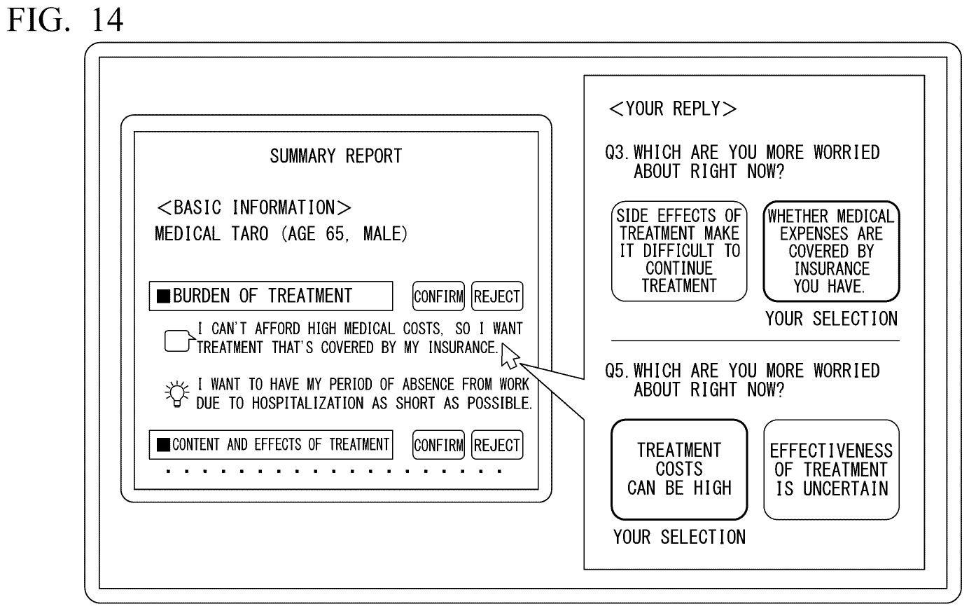

FIG. 14 is a diagram which shows another example of the GUI screen of the terminal device 10. The summary report displayed on the GUI screen may be displayed in a manner that makes it possible to distinguish whether the content is based on a specific reply of the patient's own or is an estimation. For example, it may be displayed as an icon, and when the mouse is moved over a relevant part, the corresponding specific reply of the patient own is displayed.

According to the embodiment described above, the processing circuit 120 of the information processing device 100 acquires a reply of the patient and estimates preferences of the patient and rating regarding medical treatment on the basis of the reply. The processing circuit 120 determines a response to the reply of the patient on the basis of the preferences and rating. Specifically, the processing circuit 120 determines, as a response to the reply of the patient, at least one of a narrowed response, which is a response made to the reply of the patient to narrow down a plurality of preferences, and a summary report including an approval request to the patient for each of the plurality of preferences. The processing circuit 120 then transmits the response to the reply of the patient to the terminal device 10 via the communication interface 111, or displays it on the display 113a. This makes it possible to efficiently ask questions of a patient to know the preferences of the patient regarding medical treatment without involvement of a medical professional. In particular, even when a large amount of information needs to be collected by asking questions to the patient, it is possible to ask questions efficiently to the patient without the involvement of a medical professional.

### Other Embodiment

Other embodiments will be described below. In the embodiment described above, the ratings for the preferences of a target patient is estimated using a matrix factorization that has been optimized in advance (pre-trained), but the present invention is not limited to this. For example, instead of matrix factorization, preferences and their ratings may be estimated using a large-scale language model such as chat generative pre-trained transformer (ChatGPT). In addition, in the embodiment described above, a response to the patient is determined using reinforcement learning (for example, DQN), but the present invention is not limited to this. For example, instead of reinforcement learning, the response to the patient may be determined using a large-scale language model.

When the large-scale language model is used, information output by the large-scale language model is provided to the patient in a state where it is processed using a natural language and converted into a plain text (a character string) that the patient can recognize. Next, preprocessing is performed to convert a plain text input from the patient into an input format to be input to the estimation function 122 through natural language processing. Then, the natural language processing is performed in the estimation function 122. In this case, the natural language processing model may be obtained by additionally learning a data set that was actually input to or output from the estimation function 122 for a general-purpose model.

Although several embodiments have been described, these embodiments are presented as examples and are not intended to limit a scope of the invention. These embodiments can be implemented in various other forms, and various omissions, substitutions, and modifications can be made within a range not departing from the gist of the invention. These embodiments and their modifications are included in the scope of the invention and its equivalents as described in the claims, as well as the scope and gist of the invention.

What is claimed is:

1. An information processing device comprising:
a processing circuit configured to acquire a reply from a user,
estimate medical treatment information including a plurality of preferences of the user regarding medical treatment on the basis of the reply,
determine a response to the reply on the basis of the medical treatment information, and
output the response via an output interface,
wherein the processing circuit determines, as the response, at least one of a narrowed response, which is a response made to the reply from the user to narrow down the plurality of preferences, and an approval request to the user for each of the plurality of preferences.

2. The information processing device according to claim 1,
wherein, when a series of dialogues in which the user replies to the response is repeated, the processing circuit determines the response to the most recent reply on the basis of a plurality of replies acquired in the repeated dialogues.

3. The information processing device according to claim 2,
wherein the processing circuit determines the response to the most recent reply on the basis of an attribute of the user in addition to the plurality of replies.

4. The information processing device according to claim 3,
wherein the processing circuit determines the response to the most recent reply to be either the narrowed response or an approval request to the user on the basis of the attribute of the user and at least one of a reply of the user to the narrowed response and a reply of the user to the approval request to the user.

5. The information processing device according to claim 1,
wherein the processing circuit sets as the response a summary report in which the plurality of preferences are listed and in which an object is disposed that allows the user to select whether to approve some or all of the plurality of preferences.

6. The information processing device according to claim 5,
wherein the processing circuit acquires an operation of the user on the object disposed in the summary report as a reply of the user to the approval request to the user.

7. The information processing device according to claim 5,
wherein the processing circuit, when the medical treatment information is estimated, calculates a predetermined index for each of the plurality of preferences, and
selects matters to be included in the summary report from among the plurality of preferences on the basis of the predetermined index for each of the plurality of preferences.

8. The information processing device according to claim 7,
wherein the plurality of preferences have a hierarchical structure, and
the processing circuit selects preferences to be included in the summary report from among the plurality of preferences on the basis of the predetermined index for each of the plurality of preferences and the hierarchical structure.

9. The information processing device according to claim 1,
wherein the plurality of preferences have a hierarchical structure, and
the processing circuit selects the two preferences on the basis of the hierarchical structure when the narrowed response includes a comparison of at least two preferences among the plurality of preferences.

10. An information processing method is an information processing method using a computer, comprising:
acquiring a reply from a user;
estimating medical treatment information including a plurality of preferences of the user regarding medical treatment on the basis of the reply;
determining a response to the reply on the basis of the medical treatment information;
outputting the response via an output interface; and
determining, as the response, at least one of a narrowed response, which is a response made to the reply from the user to narrow down the plurality of preferences, and an approval request to the user for each of the plurality of preferences.

11. A computer-readable non-transitory storage medium that has stored a program for causing a computer to execute
acquiring a reply from a user;
estimating medical treatment information including a plurality of preferences of the user regarding medical treatment on the basis of the reply;
determining a response to the reply on the basis of the medical treatment information;
outputting the response via an output interface; and
determining, as the response, at least one of a narrowed response, which is a response made to the reply from the user to narrow down the plurality of preferences, and an approval request to the user for each of the plurality of preferences.
